(19) United States
(12) Patent Application Publication
(10) Pub. No.: US 2014/0276502 A1
(43) Pub. Date: Sep. 18, 2014

(54) Retention Cuff for Bowel Management System

(71) Applicant: Hollister Incorporated, Libertyville, IL (US)

(72) Inventors: George J. Cisko, Spring Grove, IL (US); Thomas H. Gilman, Spring Grove, IL (US); Malford Eugene Cullum, Grayslake, IL (US); Richard I. Murahata, Libertyville, IL (US)

(73) Assignee: Hollister Incorporated, Libertyville, IL (US)

(21) Appl. No.: 14/213,666

(22) Filed: Mar. 14, 2014

Related U.S. Application Data
(60) Provisional application No. 61/917,201, filed on Dec. 17, 2013.
(60) Provisional application No. 61/790,162, filed on Mar. 15, 2013.

(51) Int. Cl. A61F 5/44

(52) U.S. Cl. CPC A61F 5/4408
USPC 604/355

(57) ABSTRACT

A retention cuff for a fecal collection device has an inflatable body with a proximal end, a proximal end flow opening, a free end, and a free end flow opening. An interior volume within the body is fillable with a medium to inflate the cuff. An axial flow path extends along an axis of and through the body and is in flow communication with the proximal and free end openings. A plurality of lateral flow pathways in the body are in flow communication with the axial flow path. The plurality of lateral flow pathways provides fluid flow in a direction different from the axial flow path.

### RELATED APPLICATION DATA

This patent is related to and claims priority benefit of U.S. provisional application Ser. No. 61/917,201 filed Dec. 17, 2013 and U.S. provisional application Ser. No. 61/790,162 filed Mar. 15, 2013, each having the same title as the instant application. The entire content of these prior filed provisional applications is hereby incorporated herein by reference.

### BACKGROUND OF THE DISCLOSURE

1. Field of the Disclosure

The present disclosure is generally directed bowel management systems, and more particularly to an irregular shaped inflatable retention cuff or balloon for a bowel drainage tube or a bowel catheter for same.

2. Description of Related Art

Commercially available drainage tubes for bowel management systems and fecal collection devices are known to include a generally spheroidal or spherical retention cuff or balloon at the free, patient insertion end of the tube. During use, these retention cuffs are also known to leak. Such retention cuffs are typically filled or inflated to a turgid or substantially fixed shape. These prior know balloon shapes are not designed to conform to the human anatomy to the extent that the device can account for the “anorectal angle” of a patient. In other words, no known devices take into account, in their design configuration, the angle created by the kink in the lower gastro-intestinal tract that prevents incontinence in an upright walking person.

Also, as a patient's anal muscles tense and relax, and as the patient moves, the shape, configuration, and contour of the patient's anal canal, rectal vault, and/or rectal ampulla changes. The typical spheroidal retention cuff does not hold a tight seal during all such conditions. Thus, the cuff can leak and the catheter can migrate in and out, and even can result in frequent expulsion of the catheter from the patient's anus.

Adding additional water or fluid to a conventional retention cuff on a catheter is a very common trouble shooting technique to help reduce leaking and to aid in retention of the catheter, i.e., to help reduce the frequency of catheter expulsion from the patient's anus. However, adding more fluid to the retention cuff often does not reduce leaking or expulsion frequency but does cause discomfort and even injury to the patient. Others have tried to solve these problems by adding less, not more, liquid to the conventional retention cuff. This does not aid in cuff retention or in preventing leaks and can increase the risk of the central opening through the balloon becoming occluded. Still others have tried to alleviate these problems by adding air instead of liquid to the retention cuff, but to the same result. These solutions have proven less than adequate in solving the foregoing problems.

Bowel catheters and drainage tubes provide a conduit to control fecal material exiting a patient's body. These devices keep material away from the skin and separate from the external environment and instead direct it to a collection bag. The conduit is held in place inside the rectum by the retention structure or cuff that is inflated after it is inserted into the rectum. The retention cuff typically includes a very flexible inflation balloon like element that is attached to an annulus. The opening in the annulus is the beginning of the conduit for fecal material. In some product forms, the annulus is simply the distal end of the conduit tubing.

One advantage is achieved by having a separate annulus in the area of balloon attachment, where this annulus is stiffer than the conduit tubing. Such a construction provides more resistance to spontaneous expulsion of the bowel catheter from the rectum. This type of spontaneous expulsion is quite undesirable. One disadvantage of providing a stiff annulus, however, is that depending on the design, the edges of the annulus can create points of stress concentration in the device. These points or edges can potentially cause irritation or damage to the patient's body where these edges come into contact with the rectal wall.

Another problem with these prior known retention cuffs is that in part, due to the anorectal angle, the rectal wall can partially occlude the opening of the annulus during use of the product. Heretofore, there has been no elegant or adequate solution to this problem.

### SUMMARY

In one example according to the teachings of the present disclosure, a retention cuff for a fecal collection device has a body with a proximal end, a proximal end flow opening, a free end, and a free end flow opening. The body is inflatable and has an interior volume that is fillable with a medium. An axial flow path extends along an axis of and through the body and is in flow communication with the proximal and free end openings. A plurality of lateral flow pathways in the body are in flow communication with the axial flow path. The plurality of lateral flow pathways provides fluid flow in a direction different from the axial flow path.

In one example, the body can have a neck at the proximal end and the axial flow path can extend through the neck.

In one example, the plurality of lateral flow pathways can be formed as separate openings or sub-channels in a side wall of the body.

In one example, the plurality of lateral flow pathways can be formed as openings or gaps between and interspersed among a plurality of lobes on the free end of the body.

In one example, the interior volume can be filled with a medium to a less than fully expanded state whereby the body is in a floppy or flaccid condition.

In one example, the retention cuff can have an annulus connected to a bowel catheter, the annulus being stiffer than the retention cuff. The annulus can have one end positioned within and along the axial flow path and in fluid communication with the axial flow path and the plurality of lateral flow pathways.

In one example, the body can have a plurality of lobes on the free end. The lobes can be spaced apart circumferentially around the retention cuff.

In one example, the lateral flow pathways can be sub-channels created by spaces between a plurality of lobes on the body.

In one example, the body can have a side wall between a toroidal section at the proximal end and the free end.

In one example, the lateral flow pathways can be formed through portions of a side wall on the body.

In one example, the free end of the body can have an irregular in shape that forms the plurality of lateral flow pathways.

In one example, the lateral flow pathways can define a flow direction that is generally perpendicular to the axial flow path.

In one example, the retention cuff can have a toroidal section on the proximal end of the body. The toroidal section can define the proximal end opening.

In one example, the retention cuff can have a plurality of spars or lobes extending axially from a toroidal section at the proximal end toward the free end of the body. The plurality of lateral flow pathways can be formed by spaces between adjacent spars of the plurality of spars or lobes.

In one example, the retention cuff can have a first toroidal section at the proximal end, a plurality of spars extending axially from the first toroidal section, and a second toroidal section at the free end of the body and connected to the plurality of spars. The first toroidal sections, plurality of spars or lobes, and second toroidal section together can define the fillable interior volume within the body.

In one example according to the teachings of the present disclosure, a fecal collection device has a bowel catheter with a patient proximal end, a patient distal end, and an internal drainage lumen. The device has a retention cuff with a body having a proximal end coupled to the patient proximal end of the bowel catheter, a proximal end flow opening in flow communication with the drainage lumen, a free end, a free end flow opening, an interior volume within the body fillable with a medium, an axial flow path extending along an axis of and through the body and in communication with the proximal and free end openings, and a plurality of lateral flow pathways in the body. The plurality of lateral flow pathways is in flow communication with the axial flow path and provides fluid flow in a direction different from the axial flow path.

In one example, the lateral flow pathways can define a flow direction that is generally perpendicular to the axial flow path.

In one example, the body can have a plurality of lobes on the free end. The lobes can be spaced apart circumferentially around the retention cuff.

In one example, the lateral flow pathways can be sub-channels created by spaces between a plurality of lobes on the body.

In one example, the retention cuff can have an annulus with one end connected to the patient proximal end of the bowel catheter and an opposite end connected to the proximal end of the retention cuff. The annulus can be stiffer than the retention cuff and can have the opposite end positioned within and along the axial flow path and in fluid communication with the axial flow path and the plurality of lateral flow pathways.

### BRIEF DESCRIPTION OF THE DRAWINGS

Objects, features, and advantages of the present disclosure will become apparent upon reading the following description in conjunction with the drawing figures, in which:

FIG. 1 shows a perspective view of a somewhat generic fecal collection device that incorporates a retention cuff or rectal catheter balloon constructed in accordance with the teachings of the present disclosure.

FIG. 2 shows a cross section taken along line 2-2 of the retention cuff or rectal catheter balloon portion of the device of FIG. 1.

FIG. 3 shows a larger end face perspective view of the retention cuff or rectal catheter balloon of FIGS. 1 and 2.

FIG. 4 shows an annulus end perspective view of the retention cuff or rectal catheter balloon of FIG. 3.

FIG. 5 shows a direct end face view of the retention cuff or rectal catheter balloon of FIG. 3.

FIG. 6 shows a direct side view of the retention cuff or rectal catheter balloon of FIG. 3.

FIG. 7 shows a perspective view of another example of a retention cuff or rectal catheter balloon configuration constructed in accordance with the teachings of the present disclosure.

FIG. 8 shows a perspective view of another example of a retention cuff or rectal catheter balloon configuration constructed in accordance with the teachings of the present disclosure.

### DETAILED DESCRIPTION OF THE DISCLOSURE

Examples of a retention cuff or rectal catheter balloon are disclosed herein and each is hereinafter referred to as a retention cuff. The disclosed retention cuffs, when filled properly, are flaccid or somewhat “floppy” rather than turgid or stiff. Prior known balloons or cuffs are turgid when filled. The present disclosure is also for a retention cuff having an irregular shape with lateral flow sub-channels, pathways, openings, or the like. The disclosed retention cuff embodiments solve or improve upon one or more of the above-noted and/or other problems and disadvantages with prior known retention cuffs and rectal catheter systems.

The disclosed retention cuffs provide fluid filled space that is a cushion between the stiff annulus edges of the drainage tube or bowel catheter and the rectal tissue of a patient. The disclosed retention cuff also provides openings, sub-channels, flow pathways, or the like in the inflated balloon or cuff that are generally perpendicular to the opening in the annulus. Such elements are provided in a portion of the balloon or cuff that is distal to the annulus so that, even when the annulus might be partially occluded, a path for fluid flow into the annulus is maintained through at least part of the retention cuff or balloon.

The disclosed retention cuff examples can adhere or cling to the walls and floor of the rectal vault. This prevents leaks by maintaining a seal therebetween, even with the action of contracting and relaxing of the muscles that control defecation and patient movement. The retention cuffs can have an ultrathin design with multiple lateral flow sub-channels that flow to the annulus. In one example, the disclosed retention cuff (not shown herein in the drawings) can include separate sub-channels that are arranged in a motif like a whiffle ball. In another example, the disclosed retention cuff can include a head and/or end face with lobes that define flow openings or pathways therebetween. Such a design can resemble the top of a castle tower or a Rook chess piece.

Turning now to the drawings, FIG. 1 shows a relatively generic example of a fecal collection device 10 for a bowel management system. The device 10 has a catheter 12 or collection tube with a patient proximal end 14 and a patient distal end 16. A retention cuff 20 or balloon, which is inflatable, is coupled to the patient proximal end 14 of the catheter 12 by a soft trans-sphincter tube section 15. The retention cuff 20 is constructed according to the teachings of the present disclosure and is described in more detail below. A bag connector assembly 22 is mounted at the patient distal end 16 of the catheter 12. As is known in the art, the bag connector assembly 22 is configured to connect the catheter 12 to a fecal collection bag (not shown). The drainage tube can be formed as a single unitary tube structure from one contiguous material or can be formed of two or more sections joined to one another. Each tube section can be formed of a different material or from the same material as one or more of the other tube sections. For example, the catheter 12 is coupled to the softer trans-sphincter tube section 15 forming a two part drainage tube.

As shown in FIG. 2, the patient proximal end 14 of the catheter 12 has a patient proximal opening that, when positioned for normal use, is in flow communication with the rectum of a patient through the retention cuff 20. As noted above, an annulus 24 can be connected to the retention cuff 20 spaced from the patient proximal end 14 of the catheter 12. The one end of the annulus 24 can define a patient proximal opening 26 (see FIG. 2) of the drainage tube. The other end of the annulus 24 can be adjacent and within a neck 28 of the retention cuff 20. The annulus 24 can be formed of a material having a durometer that is hard enough or high enough so that the annulus maintains a sufficient opening in communication with the patient proximal end 14 of the catheter 12 in order to avoid collapse and subsequent blocking of fecal outflow from the patient. One example of a suitable material is silicone rubber, 80 SH polydimethylsiloxane and fumed silica. An optimal durometer range may be between 50 Shore A and 90 Shore A hardness. The proximal opening 26 of the annulus 24 should be kept open in order to effectively receive fecal material entering the catheter 12. A relatively large lumen or flow passage 30 runs longitudinally within and along the catheter 12 and is defined by the wall material of the catheter. The lumen 30 receives effluent or fecal material from the patient's rectum.

The device 10 of FIG. 1 also is depicted as having three ports (for example, luer-style connectors), which are connectable to various lumens of a multi-lumen element member 32 connected to the catheter 12. A first or central port 34 can communicate with a first lumen (not shown) of the multi-lumen element 32. The first port 34 can be used for connecting a syringe or other infusion device in order to infuse irrigants or medications into the patient's rectum, as is known in the art. An optional or second port 36 can communicate with a second lumen (not shown) of the multi-lumen element 36 for inflation and deflation of an optional intralumenal balloon (not shown), which is also known in the art. A third or inflation port 38 communicates with a third lumen 40 (see FIG. 2) of the multi-lumen element 32 in order to inflate and deflate the retention cuff 20 with a syringe or other appropriate device, also as is known in the art.

The connector assembly 22 of the device 10 has a connector 42 that is provided at the patient distal end 16 and adapted for connection to a collection bag (not shown). The connector assembly 22 also has a plug or stopper 44 tethered to the connector 42. The plug or stopper 44 can be used to close a flow opening in the connector 42 to prevent leakage when the bag is detached, emptied, and/or replaced. The catheter 12 can also have an optional sampling or flushing port 46 positioned along the catheter a convenient point between the two ends 14, 16. The port 46 can provide access to the lumen 30 of the catheter 20 for taking fecal samples or for flushing to clean the catheter 12. In order to prevent bacterial contamination of the catheter 12, to reduce odor emanating from the device 10, and to reduce fecal build up in the catheter, frequent flushing through the optional sampling or flushing port 46 may be preferred. The port 46 should include a closable cover or cap to close off the port when not in use.

With respect to the retention cuff aspect of the disclosure, the neck 28 of the retention cuff 20 can be provided with a lower end that, during use, is connected to the trans-sphincter section 15 in this example and is in fluid communication with the lumen 30 of the catheter 12 (see FIG. 2). Though not shown herein, in one generic example, a retention cuff in accordance with the teachings of the present disclosure can have an irregular shape and contour, instead of the conventional spheroid shape. Such an irregular shaped cuff can have a whiffle-ball like structure with multiple sub-channels or flow orifices formed through the balloon shape of the cuff. Each sub-channel can be in separate fluid communication with the neck and annulus. During use, the cuff body, which is a sealed volume, will be filled to a less-than-full condition so that it is inflated but not stiff or turgid. Thus, the body shape of the filled cuff can adjust, change, and adapt upon contact with surfaces within a patient's rectal vault as those surfaces move and change.

Further, even if part of the irregular shaped retention cuff were to succumb to pressure and occlude one of the sub-channels, the other sub-channels will remain open and flowing. Thus, such an irregular shaped retention cuff can aid in preventing complete occlusion of the catheter during use, even as a patient moves and shifts position. In such an example, the sub-channels are separate passages formed through a complex shaped balloon body. The body would have an irregular or contoured side wall shape and end face shape to aid in these functions.

FIGS. 2-6 show one particular example of an irregular shaped retention cuff constructed in accordance with the teachings of the present disclosure, which is the retention cuff 20 depicted in FIGS. 1 and 2. The retention cuff 20 can be a flexible, inflatable balloon type of structure attached to an end of the relatively soft trans-sphincter section 15. The annulus 24 is a relatively short, cylindrical tube section that can be attached to one end of the trans-sphincter section 15 but within the neck 28 or retention cuff 20. In this example, the annulus 24 is connected to the interior of the retention cuff 24 but only adjacent the end of the section 15. The geometry of the retention cuff 20 and the annulus 24 is such that all edges of the annulus are protected from contact with the rectal wall of a patient by inflatable portions of the retention cuff (see FIG. 2).

In one embodiment, the inflated balloon or retention cuff 20 completely encompasses the annulus 24 and is shaped to provide openings or flow paths for effluent flow. All but one of the flow paths defines an effluent flow direction that is generally perpendicular to the path through the catheter 12, trans-sphincter section 15, annulus 24, and proximal end opening 26 of the annulus. In this example, one end of the trans-sphincter section 15 is connected to the patient proximal end 14 of the catheter 12. The other end of the section 15 is connected to the free end of the neck 28 of the retention cuff 20. The other end of the neck 28 is either joined or connected to or integrally formed as part of a toroidal section or annulus end 48 of a head or body 50 of the retention cuff 20. The annulus end 48 of the body 50 is larger in diameter than the neck 28 and can be larger than a traditional spheroid shaped retention cuff. The annulus 24 in this example is in fluid communication with and partly defines an axial flow path 52 within the body of the retention cuff 20. The axial flow path 52 is defined by a central axial opening 54 that runs lengthwise or axially through the body 50 of the inflated retention 20 cuff. The body 50, when attached to the device 10 as shown in FIG. 2, defines a sealed interior volume or space 56. The space 56 can be filled with a medium such as water or air. The annulus 24 is concentric with the neck 28 of the body 50 and opens into the axial flow path 52, which flow communicates with the axial opening 54 into the body.

The body 50 has a tapered outer side wall 60 that transitions from the smaller diameter of the neck 28 to the toroidal section 48 in a direction away from the neck. The outer side wall 60 is outwardly curved away from an axis A of the body 50 and moving away from the neck 28, as shown in FIGS. 2-5, such that the diameter of the body increases through the toroidal section 48. The outer side wall 60 continues to a free end 62 of the body 50, the free end being opposite the neck 28 on the body. The free end 62 has an irregular configuration. In this example, the body 50 is formed having a plurality of lobes 64 that are spaced radially outward from the axial opening 54 and axis A and are spaced apart circumferentially around the body. The spaced apart lobes 64 create troughs or valleys between each adjacent pair of lobes. These troughs or valleys form sub-channels 66, i.e., openings, flow pathways, or the like between the lobes 64. In this example, the body 50 has four of the sub-channels or flow pathways 66 and four of the lobes 64.

In the earlier described generic example, the sub-channels were open holes or bores that create passages through the balloon body, similar to a whiffle ball. Those passages could be in separate communication with the annulus. In this example, the sub-channels or lateral flow pathways 66 are instead defined by gaps between the spaced apart lobes 64. The sub-channels 66 are thus also separated circumferentially from one another around the circumference of the free end 62 on the body 50. The body 50 still has essentially an open end face 68 in flow communication with the axial flow path 52 and annulus 24. The opening in the end face 68 is just irregular in shape, having somewhat of an X-shape that is created by the tips of the lobes 64 and gaps (sub-channels 66) between the lobes.

With reference to FIGS. 2-6, the axial flow opening 54 is formed by an inner wall 70 of the body 50. The inner wall 70 is spaced radially inward from the outer side wall 60. The inner wall 70 is defined in part by the inward facing surfaces 72 of the lobes 64 and in part by an annular collar 74 of the body 50 below the inward facing surface of the lobes.

In this example, the effluent conduit tube or catheter 12 at its proximal end is sealed, bonded, adhered, or otherwise suitably attached to the trans-sphincter section 15, which is similarly sealed, bonded, adhered, or otherwise suitably attached to the neck 28 of the retention cuff 20. The inner wall 70 of the retention cuff is likewise sealed, bonded, adhered, or otherwise suitably attached to the exterior surface of the annulus 24. The annulus 24 is also sealed, bonded, adhered, or otherwise suitably attached to the inside surface of the neck 28 or tot eh side wall 60 adjacent the neck. The interior space or volume 56 is thus sealed to a liquid tight or air tight condition. With reference again to FIG. 2, the inflation lumen 40 opens into the space or volume 56. The flexible, balloon like structure or retention cuff 20 can be inflated with fluid injected or delivered through the inflation lumen 40. The retention cuff 20 effectively surrounds the free edge 76 of the annulus 24 and is in part situated lengthwise beyond the free edge. This provides a fluid filled space 56 between the free end 76 of the annulus 24 and any possible contact with the rectal tissue of the patient. The neck 28 of the balloon can be attached to or near the other end of the annulus 24, but in such a way that the fluid filled space will extend past the proximal end of the annulus (though not shown herein). This can protect the proximal end of the annulus from direct contact with rectal tissue. The annulus could be entirely within the dimensions of the collar 74, terminating well short of the neck within the body 50, if desired. The specific construction of these joints of the retention cuff 20 and device 10 can vary within the teachings of the present disclosure.

One advantage of the disclosed retention cuff 20 is that the free end 62 of the body 50 defines the flow pathways or sub-channels 66 between the lobes 64. The flow direction of these sub-channels 66 is generally perpendicular to the flow direction of the axial flow path 52 through the body 50 and the annulus 24. The retention cuff 20 structure can ensure free flow of effluent into the annulus 24, and also into the catheter 12 or collection tubing.

With current, known retention cuff designs, which have only a single axial flow path opening through the cuff and into the annulus, it may be that, during use, the opening into the rectal space of the patient is or becomes generally perpendicular to the opening into the annulus. In such a condition, the rectal wall can occlude the opening into the annulus, completely blocking effluent flow from the patient. With this disclosed retention cuff 20, either one or more of the lateral openings, i.e., the sub-channels or flow pathways 66 in the balloon or the central axial opening 54 into the body will generally align with the opening into the rectal space, thereby providing an unobstructed effluent flow path. This is true even where the retention cuff 20 is deformed by pressure applied by the patient's rectal wall.

A further feature of the retention cuff 20 in this example is that the balloon like structure is designed to carry the intended fluid fill volume without stretching. This minimizes or eliminates fill-induced pressure increases, and allows the flexible balloon to keep a high ability to conform to the rectal wall. In current designs, the balloon is forced to stretch to accommodate the fill volume, which reduces the balloon's ability to conform to a shape other than the inflated shape of the balloon itself.

As shown in FIGS. 1-6, the disclosed retention cuff 20 is quite different than prior known designs, which are typically spheroid, i.e., somewhat spherical or round and have a continuous rounded end face leading into the rectal catheter lumen or annulus. The disclosed retention cuff 20 can be larger in size than a conventional retention cuff. The aforementioned whiffle-ball like retention cuff configuration may perform better if larger in size than a convention cuff. However, the cuff 20 will likely perform better at even the same general size or diameter as a conventional cuff. The disclosed retention cuff 20 also has a non-spherical end face shape. In other words, the end face of the balloon does not have a consistent or continuous, bulbous surface as do prior known cuffs or balloons. The disclosed retention cuff 20 is intended to be used at less than a fully inflated condition. Thus, the cuff 20 or balloon can assume different shapes to fill and conform to a range of rectal ampulla sizes and shapes. The cuff 20 or balloon can also adapt to the changing shape of the anal canal as a patient moves. The disclosed cuff 20 or balloon can be made from an ultrathin material (because it need not be fully inflated to a turgid condition) that can more readily conform to the walls of the rectum upon being inflated or filled to permit self-sealing.

FIG. 7 shows another example of a retention cuff 80 constructed in accordance with the teachings of the present disclosure. This example is shown and described merely to show that the precise shape and configuration of the retention cuff 20 in the prior example can vary. In this example, the retention cuff 80 also has a lower end or an annulus end 82 that can connect to a separate annulus during use. The annulus end 82 can again include a neck (not shown) if desired. The retention cuff 80 can be connected to and in fluid communication with the effluent collection lumen 30 of a bowel catheter 12. The retention cuff 80 has a body 84 with an irregular shape that is very similar to that of the retention cuff 20 in FIGS. 1-6 and has multiple sub-channels or flow paths 86 defined between multiple lobes 88 at the free end 90 of the body. An axial opening 92 into the end face of the body is defined between the lobes 88. The axial opening 92 communicates with an axial flow path 94 within and along the body 84. Each lateral flow path or sub-channel 86 between the lobes 88 is in lateral fluid communication with the axial flow path 94. During use, the body 84 of the retention cuff 80, which has a sealed volume or space, will be filled to a less-than-full condition so that it is inflated but not stiff or turgid. Thus, the body shape of the filled retention cuff 80 can adjust, change, and adapt upon contact with surfaces within a patient's rectal vault as those surfaces move and change.

With either of the retention cuffs 20 or 80, the central relatively stiff annulus 24 can aid during push insertion of the retention cuff 20 and device 10 and can provide the central exit fluid path into the catheter 12. The annulus ends 48 or 82 can be relatively short and of relatively small diameter compared to current designs, and particularly the retention cuff 80 of FIG. 7. A full toroidal section makes a good liquid seal with the rectal floor, but this is also relatively short compared to existing designs. In these examples, the lobes 64 or 88, and particularly the lobes of the retention cuff 80, can have a length sufficient to extend up from the toroidal section or annulus end 48 or 82, respectively, to contact the rectal wall opposite the rectal floor, which enhances the seal to the rectal floor.

The primary difference in this example is that the lobes 88 have a thin profile spaced apart around the balloon circumference. The thin profile of the lobes 88 allows flow of effluent transverse to the axis of the axial flow path 94 while providing the capability for the retention cuff 80 to expand more readily in directions away from the tissue contact direction. This can provide a safety advantage in the event that the retention cuff 80 is unintentionally over-inflated. The lobes 88 also have the ability to deflect away from the tissue contact direction, if the anatomy is irregular or unyielding in specific locations.

In one example, the annulus end 82 can be less than ¾ in. in height, and preferably can be about ½ in. in height. The toroidal section or annulus end 82 can also be relatively short compared to existing designs. For example, the toroidal section can be less than ¾ in. in height, and can preferably be about ½ in. in height. The outside diameter of the inflated torus of the annulus end 82 can be a conventional dimension, such as, for example, about 2 in. in diameter. The slim lobes 88 of the retention cuff 80 can be taller than the torus of the annulus end 82, such as, for example, twice as tall as the torus.

The internal balloon space of the retention cuff 80 can be inflated either with, for example, water or air. Retention balloons with a relatively tall profile, like the retention cuff 80 in this example, may create a more reliable seal to the rectal floor, and so may reduce the tendency for leakage of stool or fecal matter onto the patient's skin. However, a larger dimensioned balloon may also pose a somewhat higher risk of tissue damage, especially in rectal spaces that have anatomical defects such as scarring or varicosities. The risk of damage to the rectal wall may increases when the balloon is unintentionally overinflated by the user, which can happen on occasion in a normal use environment. Over-inflation can makes the balloon even larger, perhaps to two times larger or more than the intended or desired inflated volume. This again may increase the risk of rectal wall damage.

The retention cuff 80 in this example allows for an excellent seal to the rectal floor, while mitigating the risks posed by a large balloon. The risk posed by over-inflation is also mitigated by the provision of multiple areas that can expand away from the direction of tissue contact (i.e., expansion toward adjacent lobes 88, thus minimizing the risk of tissue damage to the patient. The risk posed by an irregular rectal wall with more or less compliant areas is also mitigated by the capability of the slim balloon lobes to deflect away from the direction of tissue contact. Another problem with conventional cuff designs is that the axial fluid flow pathway can be partially or fully blocked by contact with the rectal wall that is opposite the rectal floor. The disclosed retention cuff designs, including that of FIGS. 3 and 7, provide transverse flow pathways between the lobes that address this problem.

When using the retention cuff 80 of FIG. 7, it may be easier for the user to understand that over-inflation of this design will not lead to a better liquid seal to the rectal floor, but may instead just reduce the flow pathways between the lobes. This understanding may reduce the desire to over-inflate the balloon in response to a situation where leakage is observed.

FIG. 8 discloses another example of a retention cuff 100 constructed according to the teachings of the present disclosure. In this example, the retention cuff 100 has a body 102 with a lower toroidal section or annulus end 104 and a neck 106 protruding from the annulus end toward a catheter 12 or annulus 24 (not shown). The retention cuff 100 also has an upper toroidal section 108 at a distal or free end of the cuff. A plurality of spars 110 or lobes join and extend between the lower toroidal section 104 and the upper toroidal section 108. Lateral flow pathways or sub-channels 112 into a central or axial flow path 114 are defined between the adjacent spars 110 and between an upper edge 116 of the lower toroidal section 104 and a lower edge 118 of the upper toroidal section 108. The lower toroidal section 104, spars 110 or lobes, and upper toroidal section 108 are joined in order to together define a contiguous interior fillable volume or space (not shown), which is fillable with fluid such as air or water.

The upper toroidal section 108 has a central interior or internal diameter axial flow opening 120 that forms part of the axial flow path 114. The lower toroidal section 104 also has a central interior or internal diameter axial opening 122 that forms a part of the axial flow path 114. The axial flow path 114 could communicate with the neck 106 to deliver effluent to a catheter or annulus connected to the neck.

In one example, the lower toroidal section 104 can include an internal diameter collapse resistant ring (not shown) that defines the central or axial opening 122. The collapse resistant ring can be shorter in height than the lower toroidal section 104. In this way, a lower edge of the ring would not be exposed to contact any patient tissue during use, and an upper edge of the ring would not constrain the upper edge 116 of the lower toroidal section 104.

The disclosed retention cuff 100 of FIG. 8 can provide several advantages. The design can be twice as tall as known retention cuffs currently on the market leading bowel care products. The additional height may help with leakage performance in the same manner as the above mentioned lengthier lobes 88 of the retention cuff 80. Because the upper toroidal section 108 and spars 110 are all inflated and are made of the flexible cuff material, the retention cuff 100 can deform easily to conform to the rectal vault space. The lateral openings or sub-channels 112 between the spars 110 and the upper and lower toroidal sections 108, 104 allow drainage flow perpendicular to the central drainage pathway 114, as with the earlier described embodiments. This may give more reliable drainage and, thus, better leakage performance. Also, the retention cuff of FIG. 8 may provide some over-inflation protection because, again, there are multiple areas where the cuff can expand internally, without expanding outwardly against the patient tissue interface.

The retention cuff 100 of FIG. 8 can be fabricated as follows. First, a hollow, frusto-conical dip mandrel can be created that forms the general exterior shape of the cuff, minus the lateral openings in the side wall. A cuff can be dipped, such as for example, from castable urethane, creating a double wall tapered cylinder. Then the side openings and spars can be created by sealing the double walls of the material to itself in the area of the desired openings. Then the material within these areas can be cut out to create the now sealed spars and the openings between them.

All of the above-disclosed balloons or retention cuffs can also be configured to permit two different ranges of compliance by filling the balloon with either a compressible medium (air) or a non-compressible medium (water). The lateral flow paths to the annulus are open through the several sub-channels or side openings at or near the end face of the disclosed retention cuffs so that the annulus is never occluded by the rectal ampulla wall. If one of the sub-channels is occluded, the others will still be free flowing.

The disclosed retention cuff shapes will be distinctive in the marketplace and provide differentiation from competitive products simply by the irregular appearance of the retention cuff shape. The disclosed retention cuffs will also outperform competitive products because the cuffs will provide a much better seal within the anal canal and will better conform and adapt to patient movements, adapt to patient muscle contraction and relaxing, and accommodate a broader range of patient body sizes and shapes.

The disclosed retention cuffs have an amorphous balloon shape, have a larger diameter than known cuffs and balloons, and are filled to a less-than-full or slightly flaccid condition. This renders the balloon having a somewhat floppy condition when inflated or filled. This balloon condition is better and more naturally retained by and in the body of the patient as well because of the larger size and shape adaptability during use. This can eliminate the use of less than reliable external straps, adhesive patches, and the like that are commonly used to retain a bowel catheter in place in a patient. The larger volume, lower pressure, amorphous shaped retention cuff design disclosed herein results in the bowel catheter being less likely to slip up and down within the patient.

This in turn can help maintain an internal seal and avoid the retention cuff causing anal vein abrasion and bleeding. First, the retention cuff being somewhat flaccid instead of turgid allows the cuff to be deformed by the anatomical features of the rectal ampulla of the patient. Second, the retention cuff being under less internal pressure results in the cuff applying very low pressure in rectal venules and capillaries.

The fluid filled space in the disclosed cuffs at the distal end of the annulus may help with sealing against effluent flow around the outside of the catheter and the cuff. This is because the flexible balloon element will conform better to the rectal wall versus the stiffer annulus.

The retention cuff designs disclosed herein, where the filled cuff is most minimally stretched, allows for a low or zero pressure cuff when inflated. This can result in a lower device pressure against the rectal wall, better conformance (by deformation of the balloon shape) to the rectal wall, and better conformance to the rectal wall for control of leakage around the outside of the catheter.

It is believed to be novel to have a retention cuff design that provides a fluid filled space between the edges of an annulus and any possible point of contact with rectal tissue. It is also believed to be novel to provide effluent flow openings or pathways generally perpendicular to and proximal to the axial flow path and axial opening in the end of the annulus.

Endotracheal tube retention balloons or cuffs have been configured to address the same leakage problem by utilizing a lower fill pressure. The major difference is that the trachea is nearly rigid and not compliant. Increases in endotracheal tube pressure do not translate into increases in tube volume, but instead increase pressure on the walls of the trachea, which can cut off blood circulation. Tracheal cuffs with floppy or low pressure configurations have been used to obtain an adequate seal while reducing pressure on the tracheal wall. These tracheal balloons or cuffs do not have to address the major problem that is addressed by the disclosed cuffs or balloons. The rectal ampulla is nearly 100% compliant in that the region is almost entirely and very expandable, which can make it difficult to seal. This can cause migration of the retention cuff up and down, resulting in leaks and in abrasion and bleeding of the hemorrhoidal veins in the anal canal. The cuffs and balloons disclosed herein also have the irregular end face and sub-channels to permit flow through the annulus even while the cuff is being deformed. No such problem has been or need be addressed in endotracheal cuffs.

Also, some competitive products have a softer annulus, which will tend to minimize irritation and potential trauma due the ends of the annulus. However, such products will not do as well at resisting spontaneous expulsion of the retention cuff from the patient during use.

Bowel management systems and rectal catheters of the type described herein, for which the disclosed retention cuff may be useful, are disclosed in, for example, U.S. Pat. Nos. 8,323,255, 8,075,540, 7,722,583,and 7,147,627, which are incorporated herein in their entireties.

Although certain retention cuff or balloon configurations and methods of use have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A retention cuff for a fecal collection device, the retention cuff comprising:
a body having a proximal end, a proximal end flow opening, a free end, and a free end flow opening;
an interior volume within the body fillable with a medium;
an axial flow path extending along an axis of and through the body and in communication with the proximal and free end openings; and
a plurality of lateral flow pathways in the body in communication with the axial flow path,
wherein the plurality of lateral flow pathways provides fluid flow in a direction different from the axial flow path.

2. A retention cuff according to claim 1, wherein the body has a neck at the proximal end, the axial flow path extending through the neck.

3. A retention cuff according to claim 1, wherein the plurality of lateral flow pathways are formed as separate openings or sub-channels in a side wall of the body.

4. A retention cuff according to claim 1, wherein the plurality of lateral flow pathways are formed as openings or gaps between and interspersed among a plurality of lobes on the free end of the body.

5. A retention cuff according to claim 1, wherein the interior volume is filled with a medium to a less than fully expanded state whereby the body is in a floppy or flaccid condition.

6. A retention cuff according to claim 1, further comprising an annulus connected to a bowel catheter, the annulus being stiffer than the retention cuff and having one end positioned within and along the axial flow path and in fluid communication with the axial flow path and the plurality of lateral flow pathways.

7. A retention cuff according to claim 1, wherein the body has a plurality of lobes on the free end, the lobes being spaced apart circumferentially around the retention cuff.

8. A retention cuff according to claim 7, wherein the lateral flow pathways are sub-channels created by spaces between the plurality of lobes.

9. A retention cuff according to claim 1, wherein the body has a side wall between a toroidal section at the proximal end and the free end.

10. A retention cuff according to claim 9, wherein the lateral flow pathways are formed through portions of the side wall.

11. A retention cuff according to claim 1, wherein the free end of the body has an irregular in shape that forms the plurality of lateral flow pathways.

12. A retention cuff according to claim 1, wherein the lateral flow pathways define a flow direction that is generally perpendicular to the axial flow path.

13. A retention cuff according to claim 1, further comprising a toroidal section on the proximal end of the body, the toroidal section defining the proximal end opening.

14. A retention cuff according to claim 13, further comprising a plurality of spars or lobes extending axially from the toroidal section toward the free end of the body, the plurality of lateral flow pathways being formed by spaces between adjacent spars of the plurality of spars or lobes.

15. A retention cuff according to claim 14, further comprising a second toroidal section at the free end of the body and connected to the plurality of spars, the toroidal section, plurality of spars or lobes, and second toroidal section together defining the fillable interior volume within the body.

16. A fecal collection device comprising:
a bowel catheter having a patient proximal end, a patient distal end, and an internal drainage lumen; and
a retention cuff having a body with a proximal end coupled to the patient proximal end of the bowel catheter, a proximal end flow opening in flow communication with the drainage lumen, a free end, a free end flow opening, an interior volume within the body fillable with a medium, an axial flow path extending along an axis of and through the body and in communication with the proximal and free end openings, and a plurality of lateral flow pathways in the body,
wherein the plurality of lateral flow pathways is in flow communication with the axial flow path and provides fluid flow in a direction different from the axial flow path.

17. A retention cuff according to claim 16, wherein the lateral flow pathways define a flow direction that is generally perpendicular to the axial flow path.

18. A retention cuff according to claim 16, wherein the body has a plurality of lobes on the free end, the lobes being spaced apart circumferentially around the retention cuff.

19. A retention cuff according to claim 18, wherein the lateral flow pathways are sub-channels created by spaces between the plurality of lobes.

20. A retention cuff according to claim 16, further comprising an annulus with one end connected to the patient proximal end of the bowel catheter and an opposite end connected to the proximal end of the retention cuff, the annulus being stiffer than the retention cuff and having the opposite end positioned within and along the axial flow path and in fluid communication with the axial flow path and the plurality of lateral flow pathways.
